(19) United States
(12) Patent Application Publication
(10) Pub. No.: US 2015/0080647 A1
(43) Pub. Date: Mar. 19, 2015

(54) Internal Organ Plication Device and Method

(71) Applicant: Lowell Albert WETTER, San Mateo, CA (US)

(72) Inventor: Lowell Albert Wetter, San Mateo, CA (US)

(21) Appl. No.: 14/378,780

(22) PCT Filed: Feb. 14, 2013
(86) PCT No.: PCT/US2013/026198

§ 371 Date: Aug. 14, 2014

Related U.S. Application Data
(60) Provisional application No. 61/599,734, filed on Feb. 16, 2012.

(51) Int. Cl. A61F 5/00

(52) U.S. Cl. CPC A61F 5/0086; A61F 5/0089
USPC 600/37

(57) ABSTRACT

Internal organ plication systems and methods of using the same are provided. Aspects of the plication systems include a plication maintenance device configured to maintain a folded organ configuration, and a plication/deployment component configured to fold the organ and/or deploy the plication maintenance device. The systems and methods find use in a variety of applications, including gastric plication applications, e.g., for the treatment of obesity.

### CROSS REFERENCE TO RELATED APPLICATIONS

Under 35 U.S.C. §119(e), this application claims priority to the filing date of U.S. Provisional Patent Application Ser. No. 61/599,734, filed on Feb. 16, 2012, the disclosure of which application is herein incorporated by reference in its entirety.

### INTRODUCTION

Obesity is a common condition and major public health problem in developed nations, including the United States. As of 2009, more than two thirds of American adults, approximately 127 million people, were either overweight or obese. Data suggest that 300,000 Americans die prematurely from obesity-related complications each year. Many children in the United States are also either overweight or obese. Hence, the overall number of overweight Americans is expected to rise in the future. It has been estimated that obesity costs the United States approximately $100 billion annually in direct and indirect health care expenses and in lost productivity. This trend is also apparent in many other developed nations.

For adults, the body mass index (BMI) is used to determine if one is overweight or obese. A person's BMI is calculated by multiplying body weight in pounds by 703 and then dividing the total by height in inches squared. A person's BMI is expressed as kilograms per meter squared. An adult is considered overweight if his or her BMI is between 25 and 30 kg/m². Obesity is defined as possessing a BMI between 30 and 40 kg/m². A BMI greater than 30 kg/m² is associated with significant co-morbidities. Morbid obesity is defined as possessing either a body weight more than 100 pounds greater than ideal or a BMI greater than 40 kg/m². Approximately 5% of the U.S. population meets at least one of the criteria for morbid obesity. Morbid obesity is associated with many diseases and disorders including, for example: diabetes; hypertension; heart attack; stroke; dyslipidemia; sleep apnea; pickwickian syndrome; asthma; lower back and disc disease; weight-bearing osteoarthritis of the hips, knees, ankles and feet; thrombophlebitis and pulmonary emboli; intertriginous dermatitis; urinary stress incontinence; gastroesophageal reflux disease (GERD); gallstones; and sclerosis and carcinoma of the liver. In women, infertility, cancer of the uterus, and cancer of the breast are additionally associated with morbid obesity. Taken together, the diseases associated with morbid obesity markedly reduce the odds of attaining an average lifespan. The sequelae raise annual mortality rates in affected people by a factor of 10 or more.

Current treatments for obesity include diet, exercise, behavioral treatments, medications, surgery (open and laparoscopic), and endoscopic devices. New drug treatments for obesity are currently being evaluated in clinical trials. However, a high efficacy pharmaceutical treatment has not yet been developed. Further, short-term and long-term side effects of current pharmaceutical treatments often concern consumers, pharmaceutical providers, and/or their insurers. Generally, diet or drug therapy programs have been consistently disappointing, failing to bring about significant, sustained weight loss in the majority of morbidly obese people. Currently, most operations used to treat morbid obesity include gastric restrictive procedures, involving the creation of a small (e.g., 15-35 mL) upper gastric pouch that drains through a small outlet (e.g., 0.75-1.2 cm), setting in motion the body's satiety mechanism. Other surgical strategies for treating obesity include endoscopic procedures, many of which are still in development.

One procedure of interest is the gastric plication procedure. In this procedure, also known as an imbrication or infolding procedure, a fold is made in the stomach and held in place by sutures, e.g., as shown in FIGS. 1A and 1B. While acceptable results may be achieved in such procedures, there are drawbacks. For example, such methods are not standardized. As a result, the magnitude of infolding is highly variable and not controlled. Instead, the procedures are operator dependent as they rely on suture placement that varies from operator to operator and case to case. In addition, the sutures can fail by suture fracture, being too loose, too tight, misplaced, etc.

### SUMMARY

Internal organ plication systems and methods of using the same are provided, which systems and methods provide many advantages over the currently employed methods, including suture dependent plication methods, such as reviewed above. Advantages provided by embodiments of the invention include, but are not limited to: providing standardized plication protocols that yield dependable, consistent and improved results; avoidance of suture use and disadvantages associated therewith; ease of implementation; etc. The systems and methods find use in a variety of applications, including gastric plication protocols, e.g., for the treatment of obesity.

Aspects of the plication systems include a plication maintenance device configured to maintain a folded organ configuration, and a plication/deployment component configured to fold the organ and/or deploy the plication maintenance device. The plication/deployment component may include a single device or two or more different devices which operate together to provide the plication and/or deployment of the plication maintenance device. In some instances, the plication/deployment component includes two devices, e.g., an endo-organ (e.g., intraluminal) component and extra-organ (e.g., extra-lumina, serosal) component. For example, for gastric plication embodiments of the invention, the plication/deployment component of the system may include an endo-gastric tube and a plication maintenance device deployment device, which deployment device may or may not also be employed to produce one or more gastric plications, e.g., in cooperation with the endo-gastric tube.

Embodiments of the invention further include methods of producing a one or more plications in an internal organ, e.g., methods of producing one or more gastric plications. Aspects of methods include employing an organ plication system, e.g., as described above, to produce a stable plication in an internal organ. For example, gastric plication methods are provided, where an endo-gastric tube and a plication maintenance device deployment device are used to both produce one or more gastric folds and deploy a plication maintenance device which maintains the one or more folds following removal of the endo-gastric tube and deployment device from the patient.

### BRIEF DESCRIPTION OF THE DRAWINGS

FIGS. 1A and 1B provide views of a stomach following a gastric plication procedure produced entirely with sutures, according to the Prior Art.

FIGS. 2A to 2C provide views of a plication maintenance device according to an aspect of the invention.

FIG. 3 provides a view of a plication maintenance device according to an aspect of the invention.

FIGS. 4A to 4B provide views of a plication maintenance device according to an aspect of the invention.

FIGS. 5A to 5B provide views of a plication maintenance device according to an aspect of the invention.

FIG. 6 provides a view of a plication maintenance device according to an aspect of the invention.

FIG. 7 provides a view of an endo-gastric tube according to an aspect of the invention.

FIG. 8 provides a view of an endo-gastric tube according to an aspect of the invention.

FIG. 9 provides a view of an endo-gastric tube according to an aspect of the invention.

FIGS. 10A to 10C provide views of an endo-gastric tube according to an aspect of the invention.

FIGS. 11A to 11B provide views of an endo-gastric tube according to an aspect of the invention.

FIG. 12 provides a view of a delivery device according to an aspect of the invention.

FIGS. 13A to 13O provide a sequential view of a method of plicating a stomach, according to an aspect of the invention.

FIG. 14 provides in an internal view of a plicated stomach, as produced according to an embodiment of the invention.

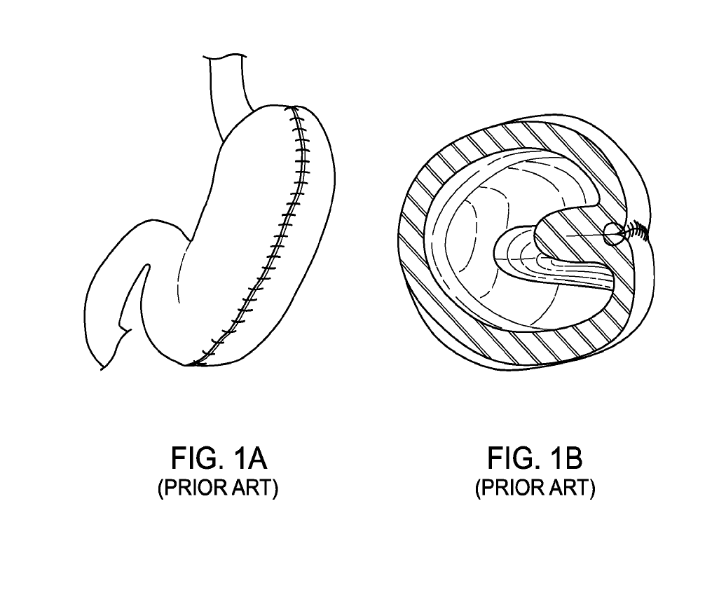

### DETAILED DESCRIPTION

Internal organ plication systems and methods of using the same are provided. Aspects of the plication systems include a plication maintenance device configured to maintain a folded organ configuration, and a plication/deployment component configured to fold the organ and/or deploy the plication maintenance device. The systems and methods find use in a variety of applications, including gastric plication applications, e.g., for the treatment of obesity.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of embodiments of the invention in greater detail, aspects of the systems and devices of various embodiments are reviewed first in greater detail, followed by a discussion of methods and kits according to certain embodiments of the invention.

### Systems and Devices

Aspects of the invention include systems and devices thereof configured for use in an internal organ plication procedure, such as a gastric plication procedure. A variety of different internal organs may be plicated (i.e., folded) using the systems and methods of the invention, including but not limited to: gastro-intestinal organs, bladders, lungs, etc. In some instances, the systems and methods are employed in gastric plication procedures. By “gastric plication” is meant one or more folds in the stomach of a patient that reduces the volume of the stomach. In certain embodiments configured to use in gastric plication procedures, the systems include a plication maintenance device, an endo-gastric tube, and a delivery device (where the tube and the delivery device may be collectively referred to herein as a plication/delivery component). Each of these components is now further described in greater detail. While the invention is now further described with specific reference to systems and methods configured for gastric plication procedures solely for purposes of ease of description, the invention is not so limited, as the systems and methods of use described below may be readily adapted to plication procedures for other organs, e.g., as described above.

### Plication Maintenance Device

As summarized above, aspects of the invention include a plication maintenance device. The term “plication maintenance device” refers to an implantable device that is configured to maintain a gastric plication. As such, the plication maintenance device is configured to contact an external (serosal) surface of the stomach and to maintain a folded gastric configuration, e.g., to achieve a reduction in stomach volume. In some embodiments, the plication maintenance device is configured to restrict expansion of the stomach along is equatorial and/or anterior-posterior axes. The magnitude of the reduction in stomach volume may vary, and in some instances may be 50% or more, such as 60% or more, such as 70% or more, such as 80%.

Plication maintenance devices in accordance with embodiments of the invention may have a variety of different configurations. In certain instances, the plication maintenance devices may include one or more planar components configured to be positioned relative to a fold in the stomach, e.g., in a fold of the stomach or across a fold in the stomach, and thereby maintain the fold in the stomach. The planar component of the device may have a wide range of length and width dimensions. In some embodiments, plication maintenance devices in accordance with embodiments of the invention have length and width dimensions that are equal or substantially equal, resulting in a plication maintenance device that has a square or substantially square component. In some embodiments, plication maintenance devices in accordance with embodiments of the invention have length dimensions that are substantially longer than the width dimensions, resulting in a plication maintenance device that is rectangular or substantially rectangular. Plication maintenance devices in accordance with embodiments of the invention may have length dimensions ranging from 1 to 10 inches and width dimensions ranging from 1 to 10 inches.

Plication maintenance devices in accordance with embodiments of the invention may have a wide range of surface areas in the planar component thereof, and are generally dimensions to provide for implantation and stable associate with the target internal organ for which they are designed, e.g., the stomach. By “surface area” is meant the total exposed area on one side of the planar component of the plication maintenance device, which, for a perfectly square or rectangular embodiment would be calculated by multiplying the length dimension by the width dimension. Plication maintenance devices according to some embodiments of the present invention have a surface area ranging from 5 to 25 square inches. In some embodiments, plication maintenance devices may have a surface area of 5 square inches or more, such as 10 square inches or more, such as 15 square inches or more, such as 20 square inches or more, such as 25 square inches.

Plication maintenance devices in accordance with embodiments of the invention may include a wide variety of geometries, including but not limited to curved or substantially curved geometries. In some embodiments, plication maintenance devices may include a curvature that roughly approximates the curvature of a stomach. In some embodiments, plication maintenance devices may include a radius of curvature that is greater than or less than the radius of curvature of a stomach. In some embodiments, plication maintenance devices may include a radius of curvature that is roughly or substantially equal to the radius of curvature of a stomach. In some embodiments, plication maintenance devices may include straight or substantially straight geometries, and may be, e.g., square or substantially square, or rectangular or substantially rectangular.

Plication maintenance devices in accordance with embodiments of the invention may have a variety of different thickness dimensions, and in some instances the thickness of a plication maintenance device may be 1 mm or more, such as 2 mm or more, such as 3 mm or more, such as 4 mm or more, such as 5 mm or more, such as 6 mm or more, such as 7 mm or more, such as 8 mm or more, such as 9 mm or more, such 10 mm or more, such as 15 mm or more, such as 20 mm or more, such as 25 mm. In some embodiments, a plication maintenance device may have a non-uniform thickness, having different thickness dimensions in different portions of the device.

Plication maintenance devices in accordance with embodiments of the invention may be configured to be cut or trimmed, either before or after forming a gastric plication, to accommodate the anatomy of a patient. For example, in some embodiments, a plication maintenance device is configured to have an initial size, and one or more portions of the plication maintenance device are configured to be trimmed or removed in order to accommodate the anatomy of the patient in whom the plication maintenance device is being used.

In some embodiments, a plication maintenance device includes one or more components that are configured to secure the plication maintenance device to the stomach, and specifically the serosal surface thereof. Such components may include, e.g., tabs, straps, flaps and the like, which are configured to secure the plication maintenance device to the external surface of the stomach. In some embodiments, these components are configured to be cut or trimmed, either before or after placement of the plication maintenance device, in order to accommodate the anatomy of the patient and to secure the plication maintenance device to the stomach.

The components that are configured to secure the plication maintenance device to the patient's stomach may be integrated with the plication maintenance device, or may be separate components that are attached to the plication maintenance device either before or after the plication maintenance device has been applied to the patient's stomach. The components may include the same material as the plication maintenance device, or may include different materials than those used in the plication maintenance device.

In some embodiments, a component that is configured to secure the plication maintenance device to the stomach may span the entire length of the plication maintenance device. In some embodiments, such a component may be configured to span less than the entire length of the plication maintenance device. In some embodiments, such components may be placed in various positions on the plication maintenance device. For example, in some embodiments, a first component may be placed at one end of the plication maintenance device and a second component may be placed at the opposite end of the plication maintenance device. In such embodiments, either of the components may be folded anteriorly or posteriorly to secure the plication maintenance device to the stomach. In some embodiments, a plication maintenance device may include three components that are configured to secure the plication maintenance device to the external surface of a stomach. In such embodiments, each of the components may be placed along the length of the plication maintenance device and configured to be folded either anteriorly or posteriorly to secure the plication maintenance device to the stomach. In some embodiments, a plication maintenance device may include four components that are configured to secure the plication maintenance device to an external surface of the stomach. In such embodiments, each of the four components may be spaced along the length of the plication maintenance device, and each component may be folded anteriorly or posteriorly to secure the plication maintenance device to the stomach. In some embodiments, two of the components may be placed in the same location along the length of the plication maintenance device and configured so that one of the components can be folded anteriorly and the other component can be folded posteriorly to secure the plication maintenance device to the stomach. Any of a variety of component configurations may be used to secure the plication maintenance device to the stomach. The embodiments discussed above are merely exemplary, and in no way limit the number or type of component configurations that can be used.

In some instances, the plication maintenance device may include component configured to be operatively connected to a deployment device, e.g., as described below. For example, the plication maintenance device may include a tubular component configured to receive a component of a deployment device (such as describe in greater detail below). This tubular component may be configured at any location of the plication maintenance device, e.g., along one edge of the plication maintenance device.

In some embodiments, a plication maintenance device may include a magnetic component. By “magnetic component” is meant a magnet or a material that is attracted to a magnet. For example, in some embodiments, the plication maintenance device includes a magnet. In some embodiments, the plication maintenance device includes a material that is attracted to a magnet, such as, e.g., a ferromagnetic material.

In some embodiments, the magnetic component is removable, meaning that the magnetic component can be associated with the plication maintenance device and separated or removed from the plication maintenance device. The magnetic component may include rigid and/or flexible elements, and in some embodiments may include a single, uniform material. In some embodiments, the magnetic component is rod-shaped.

In some embodiments, the magnetic component includes multiple magnetic components. For example, a magnetic component may include beads, rods, discs, and the like, which may be, e.g., stackable to form an extended magnetic component. These individual elements of the magnetic component may associate with one another to form a magnetic component that extends along a length of the plication maintenance device.

In some embodiments, a plication maintenance device includes magnetic components that are placed in different locations along a length of the plication maintenance device. For example, in some embodiments, the plication maintenance device includes a magnetic component that is centrally placed along a length of the plication maintenance device. In some embodiments, the plication maintenance device includes a magnetic component that is placed at a proximal end of the plication maintenance device, at a distal end of the plication maintenance device, or at both the proximal and distal ends of the plication maintenance device. In some embodiments, a plication maintenance device includes magnetic components that are spread out along a length of the plication maintenance device. In some embodiments, the magnetic components are evenly spaced along a length of the plication maintenance device. In some embodiments, the magnetic components are unevenly spaced along a length of the plication maintenance device. Any suitable combination of spacing of magnetic components may be used in plication maintenance devices according to embodiments of the invention.

A plication maintenance device in accordance with embodiments of the invention may include any of a variety of synthetic or natural materials that are suitable for use in medical implants, including polymers, fabrics, metals, and the like. In some embodiments, plication maintenance devices may include materials having any of a variety of mechanical properties, including flexible materials, rigid materials, elastic materials, and the like.

In some embodiments, a plication maintenance device includes an inert material that does not substantially interact with or elicit a response from tissues of the patient.

In some embodiments, a plication maintenance device may include a polymeric material. Non-limiting examples of suitable polymeric materials include, silicone, poly(methyl methacrylate) (PMMA), poly(2-hydroxyethyl methacrylate) (poly(HEMA), poly(methyl glutaride) (PMGI), poly(ethylene glycol dimethacrylate) (PGDMA), poly (methyl methacrylic acid)-co-PEGDMA, ethylene glycol dimethacrylate (EGDM), polyethylene, polypropylene, poly(vinyl chloride) (PVC), ethylene vinyl acetate copolymer (EVA), polycarbonate, nylon, polyurethane, phenolic resins, and the like.

In some embodiments, a plication maintenance device in accordance with embodiments of the invention includes a resorbable material. By “resorbable material” is meant a synthetic or natural material that degrades over time. Non-limiting examples of resorbable materials include resorbable polymeric materials, such as polylactide (PLA), poly-L-lactide (PLLA), poly-DL-lactide (PDLLA), polyglycolide (PGA), and various co-polymers thereof, such as a copolymer of PLA and PGA (PGLA).

Plication maintenance devices in accordance with embodiments of the invention may include a metal. Non-limiting examples of metals that may be used include alloys of nickel and titanium, such as Nitinol®, which exhibit shape memory properties. Other non-limiting examples of metals that may be used include stainless steel, titanium, tantalum, and the like.

Plication maintenance devices in accordance with embodiments of the invention may include a fabric. By “fabric” is meant a flexible material consisting of a network of natural or synthetic fibers. Non-limiting exemplary fabrics include biomedical textiles suitable for implantation in a patient.

In some embodiments, a plication maintenance device may include a woven material, a non-woven material, a knitted material, a braided material, and/or a mesh material. The term “non-uniform” as used herein is meant to indicate that a given material is not entirely homogenous, and includes, for example, knitted, woven, braided, and/or other such elements. “Non-uniform” also refers to materials that include two or more different components, such as, e.g., a mesh material including two or more different types of fibers, or including, e.g., both a fabric component and a metal component.

In some embodiments, a plication maintenance device includes a bio-active material. By “bio-active” is meant a synthetic or natural material that elicits a desired response from one or more tissues of the patient. For example, in some embodiments a plication maintenance device includes a bio-active material that facilitates integration of the plication maintenance device with the patient's tissues. Bio-active materials may be coated on the surface of the plication maintenance device or coated on the surface of one or more of the materials used to make the plication maintenance device. In some embodiments, bio-active materials may be incorporated throughout the plication maintenance device. For example, where a plication maintenance device includes a polymeric material, a bio-active material may be integrated into or distributed throughout the polymeric material.

A plication maintenance device in accordance with embodiments of the invention may include materials having a variety of surface types. For example, in some embodiments, a plication maintenance device may include materials having a rough surface. Non-limiting examples of materials having a rough surface include, e.g., Velcro® hooks and/or loops, and other such materials. For example, at least one surface of the plication maintenance device, e.g., the surface configured to touch the serosal surface of the stomach following implantation, may be configured to maintain stable association of the maintenance device with the serosal surface. For example, the surface of the device that touches the serosal surface of the stomach may be covered in hooks or analogous tissue securing structures, which physically interact with the stomach to maintain stable association. An example of a material having such hooks on one side thereof that may be employed in a plication maintenance device is the material found in the Parietex ProGrip Self-Fixating Mesh (Covidien Corporation), which material includes polylactic acid (PLA) microgrips on one surface of a physiologically compatible mesh material.

In some embodiments, a plication maintenance device may include a material having a smooth surface. Non-limiting examples of materials having a smooth surface include polymeric materials, such as, e.g., polyethylene. In some embodiments, a plication maintenance device includes a mixture of materials having both rough and smooth surfaces. For example, the material may include a first surface configured to associate with the serosal surface of the stomach, e.g., a surface with hooks or microgrips or functionally analogous tissue securing structures, and a second surface that is not associated with a serosal surface of the stomach following implantation, where the second surface is smooth, e.g., so as to reduce the occurrence of adhesions.

Plication maintenance devices in accordance with embodiments of the invention may include a surface coating. By “surface coating” is meant a material or composition that has been applied to an outer surface of the plication maintenance device or applied to the outer surface of a material used to make a plication maintenance device. Non-limiting exemplary surface coatings include surface coatings that alter the appearance, adhesion properties, wettability, corrosion resistance, wear resistance, or other physical and/or chemical properties of the surface of the plication maintenance device. Additional non-limiting examples of surface coatings include proteins, lipids, nucleic acids, polysaccharides, and the like, which may be applied to the plication maintenance device as a surface coating. Surface coatings may generally be bio-active or non-bio-active.

Plication maintenance devices in accordance with embodiments of the invention may include a surface treatment. By “surface treatment” is meant an alteration of the physical and/or chemical properties of the surface of the plication maintenance device or a material used to make the plication maintenance device. Non-limiting examples of surface treatments include alterations to the appearance, adhesion properties, wettability, corrosion resistance, wear resistance, or other physical and/or chemical properties of the surface of the plication maintenance device or a material used to make the plication maintenance device. Non-limiting examples of surface treatments include physical alteration of the surface through the application of heat, plasma treatment, or the application of chemical compounds that alter the surface of the plication maintenance device.

In some embodiments, a plication maintenance device may include a non-uniform material, such as, e.g., a mesh or braided material, wherein one of the materials includes a surface treatment and/or a surface coating, and another material does not. For example, a woven material may include a first fiber type that has been coated with a surface coating and/or subjected to a surface treatment, and a second fiber type that has not. These two types of fibers may then be woven together to form a woven material that has a partial or selective surface coating.

In some embodiments, a plication maintenance device may include a non-uniform material, such as, e.g., a mesh or braided material comprising fibers that have been subjected to different surface treatments or have had different surface coatings applied to them.

In some embodiments, a plication maintenance device may include one or more active elements that provide a particular functionality to the plication maintenance device. For example, in some embodiments, a plication maintenance device may include a gastric electrical stimulator that is configured to provide an electrical signal to the stomach. In some embodiments, a plication maintenance device may include a gastric pacemaker that is configured to modulate muscle activity in the stomach. In some embodiments, a plication maintenance device may include a neuromodulator that is configured to modulate nerve activity in the stomach. In some embodiments, a plication maintenance device may include a drug delivery component that is configured to deliver one or more drugs or active compounds to the stomach. In some embodiments, a plication maintenance device may include a component that is configured to apply a mechanical signal to the stomach. Non-limiting examples of such components include elastic components configured to apply a mechanical stress or a mechanical strain to the stomach.

In some embodiments, a plication maintenance device may include a hybrid material that is a combination of different material components described herein. For example, in some embodiments, a plication maintenance device may include a combination of metallic components and fabric components, any of which may be, e.g., bio-active. Any of a variety of combinations of materials and material modifications may be used to form a plication maintenance device for use with the disclosed systems and methods.

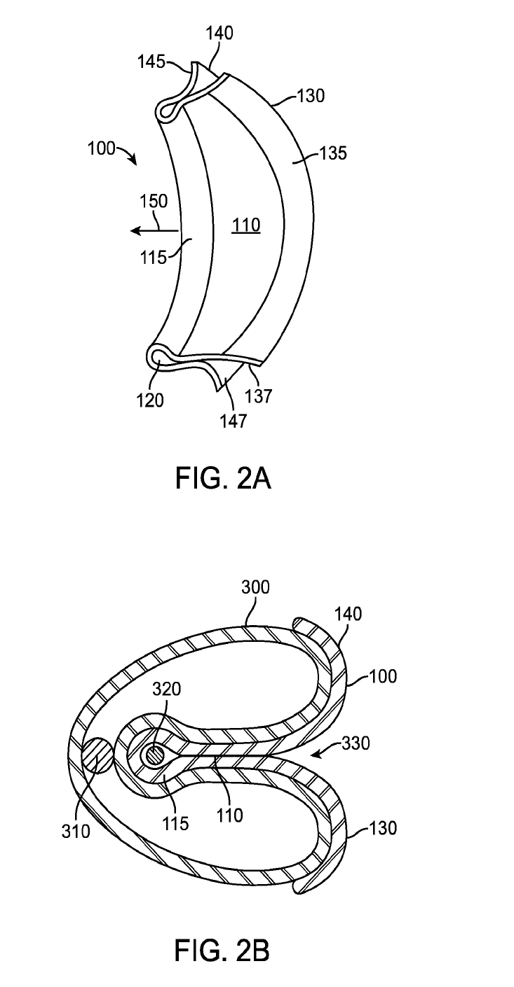

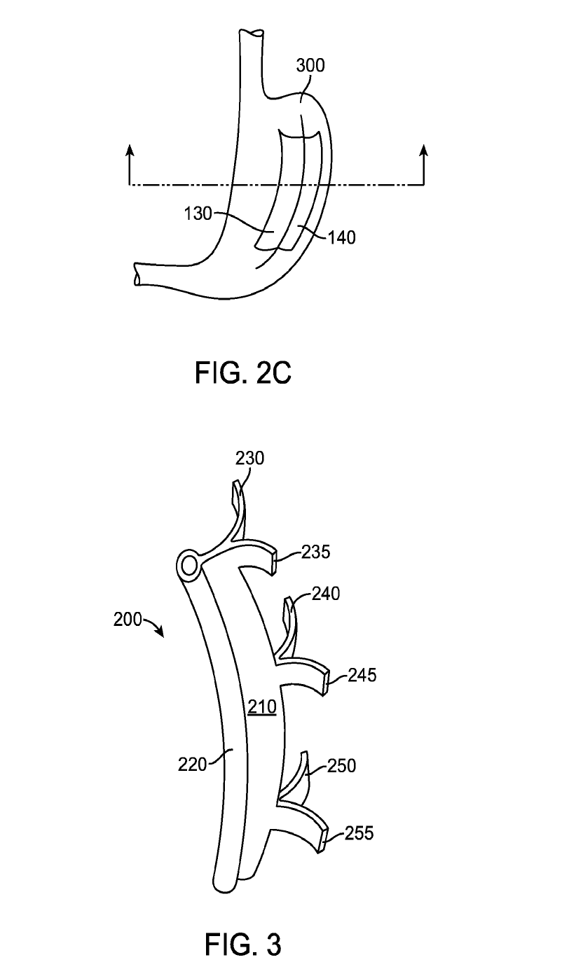

Plication maintenance devices are now further described in connection with specific embodiments illustrated in figures of the application. Referring now to FIG. 2A, an embodiment of a plication maintenance device according to the present invention is depicted. In the depicted plication maintenance device, device 100 includes a planar component 110 and a tubular component 115 positioned on one side of the planar component, which tubular component 115 includes lumen 120. The tubular component is configured to operatively interact with a deployment device (see FIG. 2B described in greater detail below) during deployment. Each side of planar component 110 may include tissue securing structures, e.g., hooks, barbs, microgrips, etc., to secure the each of the opposing surfaces of the planar component 110 to the serosal surface of the stomach in the folded region following implantation. As shown, the maintenance device includes has a curved configuration, where the radius of curvature 150 approximates that of the stomach. Also shown in FIG. 2A are first and second securing components 130 and 140 which run the length of one side of the planar component and are configured to further secure the maintenance device to the serosal surface of the stomach in a non-folded region following implantation. On first surfaces 135 and 145 of these securing components, tissue securing means, e.g., as described above, may be present. On the opposing surfaces 137 and 147 the surface may be smooth, e.g., to discourage the formation of adhesions. In the embodiment shown in FIG. 2A, the tubular and planar components may be provided using any convenient configuration. For example, the tubular and planar components may be provided by simply folding an initial structure over on itself, to provide a lumen 120, where the two layers that make up the planar component are stably adhered to each other, e.g., with an adhesive, a physical securing means, e.g., hook, barb, etc. Alternatively, a distinct tube may be attached to one side of a planar structure, which may or may not be multilayered in such an embodiment.

FIG. 2B provides a view of the maintenance device positioned in the fold of plicated stomach during a plication procedure. As seen in FIG. 2B, stomach 300 has been manipulated using endo-gastric tube 310 and a rod 320 of a delivery device to produce a fold 330. Maintenance device 100 shown in FIG. 2A is positioned so that the tube component is at the bottom of the fold and the planar component is positioned within the fold 330. Securing components 130 and 140 are attached to the serosal surface. The serosal side of the maintenance device is stably associated with the serosal surface of the stomach by tissue securing elements, e.g., as described above. FIG. 2C provides a view of the serosal surface of the stomach 300 shown in FIG. 2B.

Referring now to FIG. 3, another embodiment of a plication maintenance device according to the present invention is depicted. The depicted plication maintenance device 200 includes planar component 210 and tubular component 220, which have analogous structures and functions to the planar and tubular components 110 and 115 of the embodiment shown in FIG. 2A. However, in contrast to the embodiment depicted in FIG. 2A, the embodiment shown in FIG. 3 includes 3 pairs of tissue securing tabs, 230 & 235, 240 & 245 and 250 & 255, which pairs of tabs are equidistantly spaced along the edge of the planar component opposite the edge that includes the tubular component. Each of the tabs 230 & 235, 240 & 245 and 250 & 255 may have a first surface that includes tissue securing elements and a second surface that is smooth, e.g., as described above.

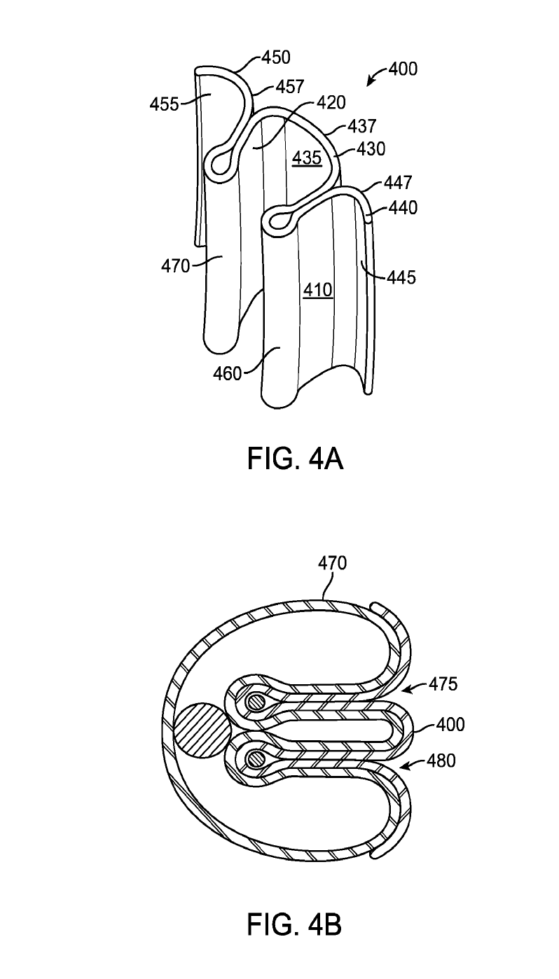

Referring now to FIG. 4A, another embodiment of a plication maintenance device according to an embodiment of the present invention is depicted. The embodiment shown is FIG. 4A is configured for use in a multi-plication procedure. As shown, device 400 includes two distinct planar components 410 and 420 joined to each other by bridging region 430. Also present are securing components 440 and 450 which run the entire length of the edges of the planar components 410 and 420, respectively. The securing components 440 and 450 include first sides 445 and 455 having tissue securing elements, e.g., microgrips, and second sides 447 and 457 that are smooth. Also shown are tubular components 460 and 470 which are configured to receive a deployment device component. FIG. 4B provides a view of the maintenance device 400 of FIG. 4A positioned in the folds of plicated stomach during a plication procedure. In FIG. 4B, two folds 475 and 480 are produced in stomach 470 via interaction of the rods of a delivery device and an endo-gastric tube, in a manner analogous to that described above in connection with FIG. 2B.

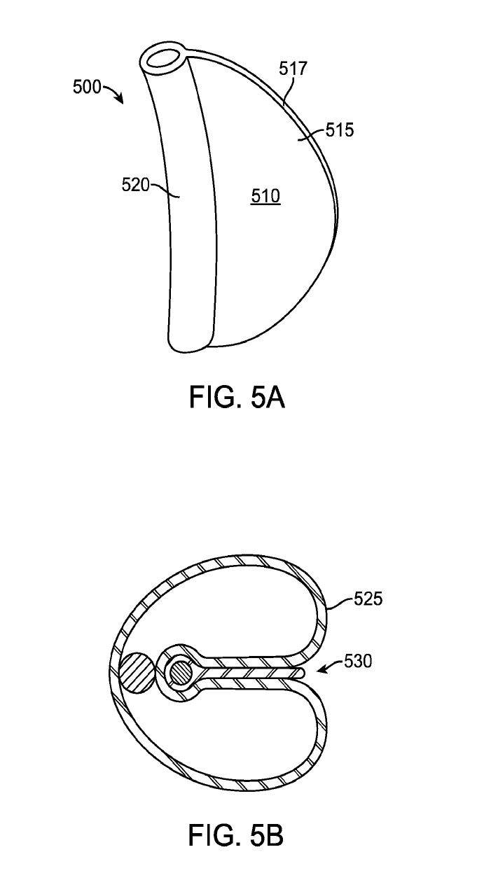

FIG. 5A depicts yet another version of a plication maintenance device. In FIG. 5A device 500 includes planar component 510 and tubular component 520, which components are analogous to the planar and tubular components described above. However, device 500 lacks any distinct tissue securing component. Instead, the device includes opposing surfaces 515 and 517 of planar component 510 which displays tissue securing elements, e.g., hooks, barbs, microgrips, e.g., as described above. FIG. 5B provides a view of the maintenance device 500 of FIG. 5A positioned in the fold of a plicated stomach during a plication procedure. In FIG. 5B, fold 530 is produced in stomach 525 via interaction of a rod of a delivery device and an endo-gastric tube, in a manner analogous to that described above in connection with FIG. 2B. As can be seen in FIG. 5B, the plication maintenance device resides substantially, if not entirely within fold 530. Where a portion of the device does not reside in the fold, that portion of the device will in some instance be 10 wt. % or less, such as 5 wt. % or less.

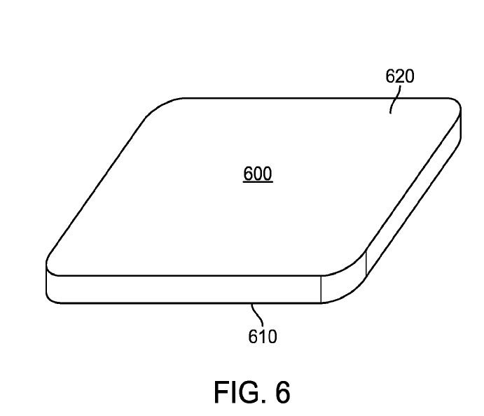

FIG. 6 depicts yet another version of a plication maintenance device 600. In the device shown in FIG. 6, device 600 includes a first surface 610 which displays tissue securing elements, e.g., hooks, barbs, microgrips, e.g., as described above. Second surface, 620, is smooth, e.g., to prevent adhesions. This embodiment is employed in protocols where the plication maintenance device is applied to the serosal surface of the stomach across the fold or folds, but no portion of the maintenance device lies inside of the folds. FIG. 100, described below, provides a view of how this type of plication maintenance device may be employed in a gastric plication procedure.

### Endo-Gastric Tube

As summarized above, systems in accordance with embodiments of the invention may include an endo-gastric tube. By “endo-gastric tube” is meant an elongated apparatus that is configured to be placed inside the stomach of a patient. As such, an endo-gastric tube is generally configured to be inserted into the mouth of a patient and to pass through the esophagus and into the patient's stomach. Endo-gastric tubes in accordance with embodiments of the invention may have any of a variety of dimensions and geometries. In some instances, endo-gastric tubes in accordance with embodiments of the invention have a circular or substantially circular cross section. However, endo-gastric tubes in accordance with embodiments of the invention are not necessarily limited to those having a specific cross-sectional geometry. For example, endo-gastric tubes in accordance with embodiments of the invention may have any of a variety of cross-sectional geometries, including those that are substantially square, substantially triangular, and the like.

Endo-gastric tubes in accordance with embodiments of the invention may include a magnetic component and/or may be configured to define a gastric volume. Endo-gastric tubes in accordance with embodiments of the invention may be used in conjunction with a plication maintenance device, as described above, to create a gastric plication that reduces the volume of a patient's stomach. In some embodiments, an endo-gastric tube is configured to be coupled to a magnetic component of a deployment device and/or plication maintenance device in order to align the plication maintenance device with the stomach and/or the endo-gastric tube. In some embodiments, an endo-gastric tube is configured to be coupled to a magnetic component of a deployment device and/or plication maintenance device in order to create and/or define a gastric plication.

Endo-gastric tubes in accordance with embodiments of the invention may include any suitable material and may have any suitable dimensions to facilitate insertion into a patient's stomach. In some embodiments, an endo-gastric tube is configured to pass through the patient's esophagus into the patient's stomach. In some embodiments, an endo-gastric tube is configured to pass beyond the pylorus of the patient's stomach and into the duodenum. Endo-gastric tubes in accordance with embodiments of the invention may have a variety of different dimensions. For example, in some embodiments, the outer diameter of an endo-gastric tube may be 10 millimeters or greater, such as 12 millimeters or greater, such as 14 millimeters or greater, such as 16 millimeters.

In some embodiments, an endo-gastric tube may be multi-luminal. In some embodiments, an endo-gastric tube may include one or more aspiration ports and/or may be configured to supply suction to the stomach so that liquid and/or gaseous contents of the stomach can be removed. For example, an endogastric tube may include a single aspiration port or more two or more aspiration ports, e.g., 3 or more, 4 or more, 5 or more, etc., aspiration ports, which port or ports may be positioned at any convenient location of the device, e.g., to provide for remove of fluid from the stomach during the plication procedure. Such ports and removal of fluid may also be employed to ensure the correct placement of the maintenance device on the serosal surface of the stomach. In some embodiments, an endo-gastric tube may be configured to inflate the patient's stomach.

In some embodiments, an endo-gastric tube may include one or more inflatable components. For example, an endo-gastric tube may include a distal inflatable balloon that can be inflated to secure the endo-gastric tube in position. In some embodiments, an endo-gastric tube is configured so that the distal inflatable balloon can be positioned beyond the pylorus of the stomach and inflated in the patient's duodenum to secure the endo-gastric tube in position. The endo-gastric tube may also include one or more inflatable components that are configured to be deployed in the stomach, e.g., to ensure a desired plication, to provide for the desired stomach volume following plication, e.g., in those instances where the desired diameter of the plicated stomach is greater than the diameter of the esophagus, etc.

Endo-gastric tubes in accordance with embodiments of the invention may include a magnetic component. By “magnetic component” is meant a magnet or a material that is attracted to a magnet. For example, in some embodiments, an endo-gastric tube includes a magnet. In some embodiments, an endo-gastric tube includes a material that is attracted to a magnet, such as, e.g., a ferromagnetic material. In some embodiments, the magnetic component of the endo-gastric tube is removable, meaning that the magnetic component can be associated with an endo-gastric tube and separated or removed from the endo-gastric tube. The magnetic component may be rigid or flexible, and in some embodiments includes a single, uniform material. In some embodiments, the magnetic component of the endo-gastric tube is rod-shaped. In some embodiments, the magnetic component of the endo-gastric tube is made of multiple magnetic components. For example, a magnetic component may include beads, rods, discs, and the like, which may be, e.g., stackable to form an extended magnetic component. The individual magnetic components may associate with one another to form a magnetic component that extends along a length of the endo-gastric tube. In some embodiments, the magnetic component of the endo-gastric tube is configured to extend along a length of the endo-gastric tube. The magnetic component may be placed in any appropriate location along the length of the endo-gastric tube in order to facilitate folding the stomach into a folded gastric configuration to form a gastric plication. Endo-gastric tubes in accordance with embodiments of the invention may include magnetic components that are placed in different locations along the endo-gastric tube. For example, in some embodiments, an endo-gastric tube includes a magnetic component that is centrally located along the endo-gastric tube. In some embodiments, the endo-gastric tube includes two or more magnetic components that may be placed proximally and distally to define a length along the endo-gastric tube. In some embodiments, multiple magnetic components are spaced along a length of the endo-gastric tube. In some embodiments, the magnetic components may be evenly spaced. In some embodiments, the magnetic components may be unevenly spaced. Any suitable combination of magnetic component spacing may be used in endo-gastric tubes according to embodiments of the invention.

Where desired, the endo-gastric tube may include steering element configured to provide proper placement of the tube. The steering elements may vary, where a number of endoscopic and catheter based steering mechanisms are known, e.g., wires, articulating mechanisms, etc. Any convenient steering mechanism may be employed.

Where desired, the endo-gastric tube may include a visualization element at one or more locations. Any convenient visualization element may be present, where visually elements of interest include optical guides, cameras, etc. As desired, a light source may also be provided. Instead of, or in addition to, a visualization element, other sensor elements may be present, e.g., temperature sensors, pH sensors, etc.

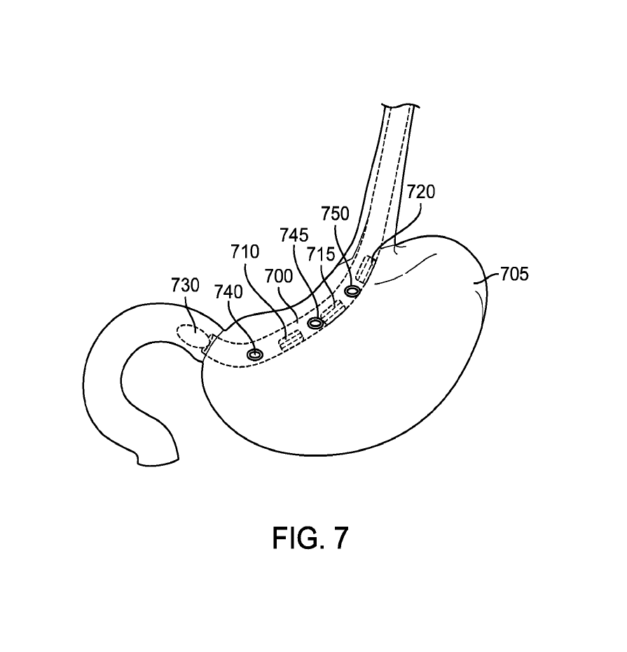

Referring now to FIG. 7, a schematic diagram of a stomach 705 is depicted, showing an endo-gastric tube 700 in accordance with an embodiment of the invention. The endo-gastric tube 700 includes multiple magnetic components 710, 715 and 720 that are spaced along a length of the endo-gastric tube 700. The endo-gastric tube 700 also includes an inflatable balloon 700 at the distal end, as well as aspiration ports 740, 745 and 750.

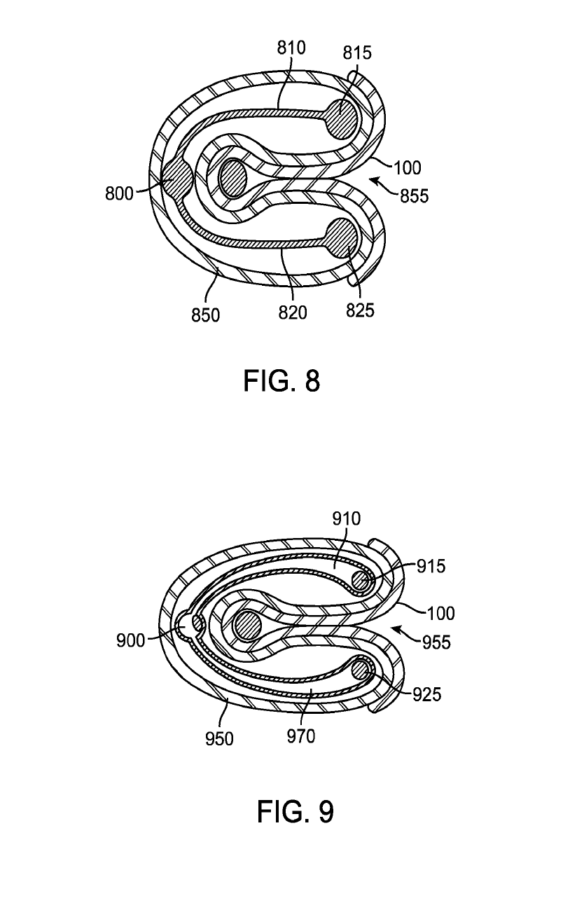

In some instances, the endo-gastric tube may include additional features which help ensure a proper plication is produced during the plication procedure. One such embodiment is illustrated in FIG. 8. As shown in FIG. 8, tube 800 includes first and second deployable arms 810 and 820, which may be deployed following insertion of the tube into the stomach in order to ensure proper formation of the fold. As shown, arms 810 and 820 include distal spherical structures 815 and 825 respectively. When employed during a plication procedure as shown, the arms and spherical structures further define the fold 855 of stomach 850 into which the plication maintenance device 100 is positioned.

A variation of this embodiment is shown in FIG. 9. In FIG. 9, tube 900 includes first and second inflate-ably deployable arms 910 and 920, which may be deployed following insertion of the tube into the stomach in order to ensure proper formation of the fold. As shown, arms 910 and 920 include distal spherical structures 915 and 925 respectively. When employed during a plication procedure as shown, the arms and spherical structures further define the fold 955 of stomach 950 into which the plication maintenance device 100 is positioned.

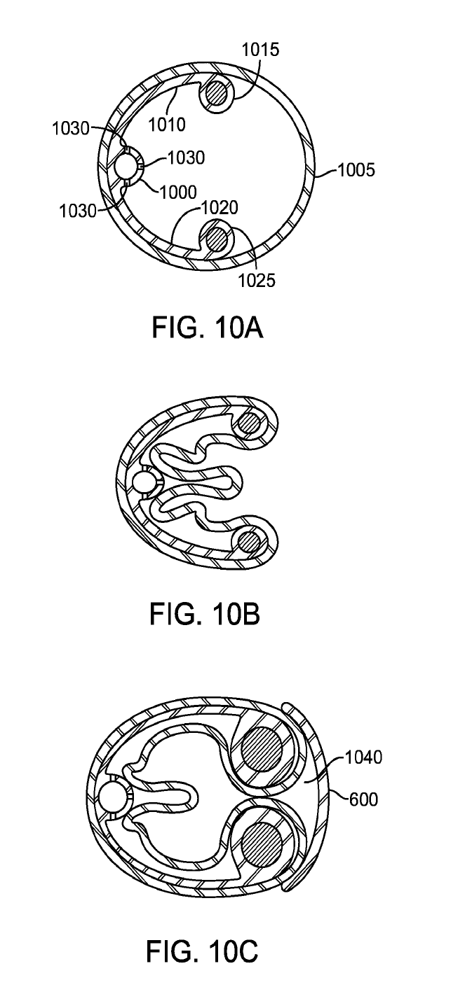

FIGS. 10A to 10C provides views of yet another embodiment of an endo-gastric tube. In FIG. 10A, tube 1000 is positioned in stomach 1005 and includes deployed arms 1010 and 1020, each with spherical structure 1015 and 1025 at the end, respectively. Also shown are three aspiration ports 1030. During a plication procedure, a vacuum is applied to the interior of the tube 1000 so as to suck fluid from the stomach and infold the stomach, e.g., as shown in FIG. 10B. The ends of the two arms are then brought together using any convenient protocol, e.g., as shown in FIG. 100. For example, the ends may be brought together manually, via mechanical manipulation of the arms, via magnetic attraction of the arms, e.g., where the elements 1015 and 1025 are magnetic, etc. Finally, as shown in FIG. 100, plication maintenance element 600 (as shown in FIG. 6) is positioned on the serosal side of the stomach across the fold 1040 to maintain the plication.

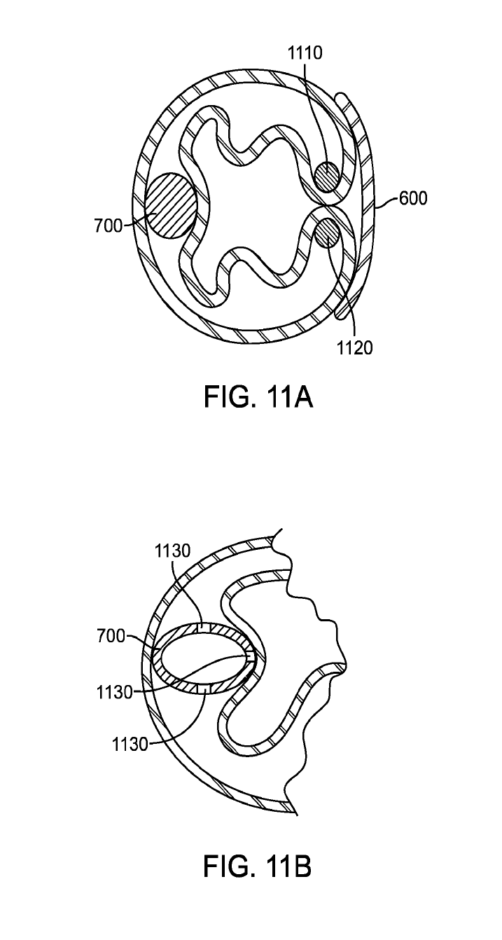

In yet another variation, the gastric tube as shown in FIG. 7 may be employed with distinct plication production elements, e.g., as shown in FIG. 11A. In FIG. 11A, but 700 is used in conjunction with distinct plication elements 1110 and 1120. During a plication procedure, elements 1110 and 1120 are brought together and then the maintenance device 600 is applied. FIG. 11B shows the aspiration ports 1130 of device 700.

### Delivery Systems

As summarized above, systems in accordance with embodiments of the invention may include a delivery system. By “delivery system” is meant one or more components that are configured to deliver a plication maintenance device to the abdominal cavity of a patient. Delivery systems in accordance with embodiments of the invention generally include all necessary components for accessing the abdominal cavity of a patient, introducing a plication maintenance device into the abdominal cavity, and deploying the plication maintenance device inside the abdominal cavity. The delivery system may also be employed in producing the one or more folds in the

Delivery systems in accordance with embodiments of the invention may include a sheath for abdominal delivery of the plication maintenance device. The sheath may include any suitable natural or synthetic material, and is generally configured to cover or encase a plication maintenance device. In some embodiments, a plication maintenance device may be folded, rolled, crumpled, compressed, constrained, or otherwise reduced in size in order to be placed inside the sheath.

In some embodiments, a plication maintenance device is mounted on a delivery system to facilitate delivery of the plication maintenance device to the patient's abdominal cavity. The plication maintenance device can be mounted on the delivery system in any suitable manner to facilitate delivery of the plication maintenance device to the patient's abdominal cavity and detachment of the plication maintenance device from the delivery system. For example, a plication maintenance device may be mounted on a delivery system using, e.g., clasps or snaps that may be undone once the plication maintenance device has been delivered to the patient's abdominal cavity. In some embodiments, a plication maintenance device may be mounted on a delivery system using magnetic components. In some embodiments, a plication maintenance device may be mounted on a delivery system using levers and/or pulleys that may be activated or controlled by a user to deploy the plication maintenance device inside the patient's abdominal cavity.

Delivery systems in accordance with embodiments of the invention may include movable components that are configured to deploy the plication maintenance device. Such delivery systems may include passive deployment elements, such as malleable and/or pre-shaped components that are configured to deploy the plication maintenance device. In some embodiments, the delivery systems include active deployment components, which may include joints, levers, pulleys, and the like, which are configured to deploy the plication maintenance device. In some embodiments, delivery systems may include control components that are configured to allow a user to control the movement and/or placement of the delivery system inside the patient's abdominal cavity. In some embodiments, the delivery systems may include control components that allow a user to control the deployment of the plication maintenance device inside the patient's abdominal cavity.

In some embodiments, the delivery devices include one or more components that are configured to support the plication maintenance device. Such components may include rods, arms, branches, or other similar structures that are configured to support the plication maintenance device once it has been deployed inside the patient's abdominal cavity. In some embodiments, the support components may include magnetic components. In some embodiments, a magnetic component of the support component is configured to interact with a magnetic component of the endo-gastric tube in order to facilitate forming a gastric plication mediated by the plication maintenance device. In some embodiments, the support components are configured to stretch and/or extend the plication maintenance device following its deployment.

In one embodiment of interest, the delivery system includes a rigid rod on which the maintenance device is mounted during deployment, where the rod not only serves to deploy the maintenance device, but also serves in producing the fold in the stomach. For example, the rod may be used during deployment to push the stomach in from the serosal side in order to produce the desired plication. The delivery system may be configured to work with the endo-gastric tube in producing the plication, e.g., by having one or more magnetic elements (or being fabricated from a magnetic material) such that the rod is attracted to the endo-gastric tube and produces the desired gastric plication.

Delivery systems in accordance with embodiments of the invention may include an outer introducer that is configured to provide access to the patient's abdominal cavity and to allow the plication maintenance device to pass through the patient's abdominal wall. The outer introducer is generally configured to have a length and a diameter that are suitable for use with abdominal and/or abdominal laparoscopic surgical procedures. In some embodiments, the outer diameter of the outer introducer device ranges from 5 to 15 millimeters. In some embodiments, the length of the outer introducer ranges from 20 to 30 centimeters.

Delivery systems in accordance with embodiments of the invention may include a trocar that is configured to create an opening in the patient's abdominal wall. In some embodiments, a delivery system includes a trocar that has a diameter and is configured to create one or more ports in the patient's abdominal wall. In some embodiments, a trocar is configured to introduce a plication maintenance device and/or a delivery device into the patient's abdominal cavity. In some embodiments, the trocar has a diameter of 5 mm or more, such as 7 mm or more, such as 10 mm or more, such as 12 mm or more, such as 15 mm.

In some embodiments, the plication maintenance device may be folded, rolled, crumpled, compressed, constrained, or otherwise reduced in size for purposes of introducing the plication maintenance device into the abdominal cavity of a patient. Once the plication maintenance device has been placed inside the patient's abdominal cavity, it can be unfurled. In some embodiments, the components of the delivery system are removed from the patient's abdominal cavity before the plication maintenance device is unfurled.

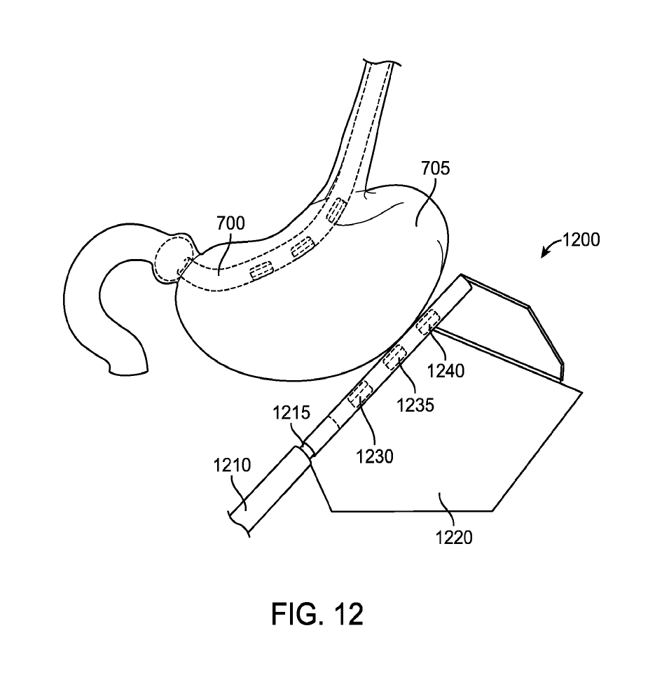

An example of a delivery system shown during a plication procedure is illustrated in FIG. 12. During a plication procedure illustrated in FIG. 12, following introduction of endo-gastric tube 700 into stomach 705 (e.g., as described in connection with FIG. 7), delivery device 1200 is employed to produce a plication and deploy the plication maintenance device. Delivery device 1200 includes delivery sheath 1210 from which is extended rod 1215. Prior to extension of the rod 1215 from sheath 1210, the maintenance device 1220 is wrapped around rod 1215 in order to fit inside of sheath 1210. As shown, rod 1215 is extended through the tubular component of maintenance device 1220. Also present on rod 1215 are magnetic elements 1230, 1235 and 1240 which are configured to match up magnetic elements on the endo-gastric tube 700 during the folding procedure. While the embodiment of the delivery device in FIG. 12 includes a single rod which is extended from a single sheath during deployment and plication, a given delivery device may include two or more distinct rods, e.g., where two or more plications in the stomach are desired, e.g., as described above in connection with the embodiment shown in FIGS. 4A and 4B.

### Methods

Methods in accordance with embodiments of the invention may be used to reduce the stomach volume of a patient by operatively coupling a plication maintenance device to the patient's stomach in a manner sufficient to produce oneo more gastric plications. In some embodiments, the methods involve placing an endo-gastric tube inside the patient's stomach and contacting the external surface of the patient's stomach with a plication maintenance device in a manner sufficient to form a gastric plication that is mediated by the interaction of the plication maintenance device with the endo-gastric tube. In some embodiments, the methods involve magnetically coupling a delivery device component and/or the plication maintenance device to the endo-gastric tube to form a gastric plication.

In some embodiments, the methods involve placing an endo-gastric tube that includes a distal inflatable balloon inside the patient's stomach and inflating the distal inflatable balloon. For example, in some embodiments, the methods involve inserting an endo-gastric tube through the patient's mouth and passing it into the stomach. In some embodiments, the distal end of the endo-gastric tube is passed beyond the pylorus and into the duodenum. In some embodiments, an inflatable balloon located on the distal end of the endo-gastric tube may be inflated to secure the distal end of the endo-gastric tube in a desired position.

In some embodiments, the methods involve creating an opening in the patient's abdomen and passing a plication maintenance device through the opening. For example, in some embodiments, an opening in the patient's abdomen may be created using a scalpel, a trocar, or any device or instrument suitable for making an incision. Once an opening in the patient's abdominal wall has been created, a plication maintenance device can be passed through the opening. In some embodiments, a plication maintenance device may be passed through the opening in the abdominal wall using a delivery device, e.g., as illustrated in FIG. 12. In such embodiments, the methods may include deploying the plication maintenance device from the delivery device inside the patient's abdomen, following by production of one or more plications in the stomach and securement of the plication(s) with the plication maintenance device.

In some instances, the plication methods described herein are sutureless, in that sutures are not employed. As such, sutures are not employed to maintain the plication in the stomach, or to secure the plication maintenance device to the stomach.

Methods in accordance with embodiments of the invention find use in, e.g., the treatment of obesity. For example, in some embodiments, methods in accordance with embodiments of the invention involve reducing the stomach volume of a patient who has been diagnosed with obesity.

Methods in accordance with embodiments of the invention may incorporate a variety of different diagnosis techniques for determining whether a patient is obese. For example, in some embodiments, an obesity diagnosis may be performed by measuring the body mass index (BMI) of a patient and comparing the measured BMI to a set of standard or control measurements. BMI may be calculated by measuring the height and mass of a patient and dividing the mass (in kg) by the height (in meters) squared to produce a BMI calculation. In some embodiments, the methods involve diagnosing a patient with obesity when the patient has a BMI of 25 kg/m2 or higher, such as 30 kg/m2 or higher, such as 35 kg/m2 or higher, such as 40 kg/m2 or higher.

In some embodiments, an obesity diagnosis may be performed by comparing a patient's height and weight to standard height and weight charts, such as gender-specific and/or age-specific height and weight charts. Such height and weight charts may be used to determine whether a patient is obese based on normal, healthy height and weight measurements for patients of similar age and/or gender.

In some embodiments, an obesity diagnosis may be performed using bioelectrical impedance analysis (BIA). BIA can be measured using a machine that passes an electrical signal through the patient's tissues and determines the amount of each of the different kinds of tissues in the patient's body, including muscle tissue, lean tissue, fatty tissue, and water. The greater the amount of fatty tissue in the patient's body, the greater resistance the electrical signal encounters. A diagnosis of obesity can be made by comparing the measured BIA to standard or control measurements from patients with normal, healthy height and weight.

In some embodiments, an obesity diagnosis may be performed using calipers that are configured to measure the amount of body fat in different parts of a patient's body. In such embodiments, the calipers are applied to the patient's body in a variety of locations, and the skinfold thickness is measured. The measurements are then compared to standard or control measurements taken from patients with normal, healthy height and weight to determine whether the patient is obese.

The description of embodiments of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms that are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In certain embodiments, the subjects, e.g., patients, are humans.

### Kits

Also provided are kits that at least include the subject systems and devices or components thereof, e.g., as described above, and instructions for how to use the devices in a procedure, such as a gastric plication procedure. In some embodiments, the kits can include a plication maintenance device configured to maintain a folded gastric configuration. In some embodiments, the kits may include a plication/deployment component, e.g., as described above, may include an endo-gastric tube and/or a delivery device. Where the kits include a delivery device, e.g., a minimally invasive delivery device, as described above, the device may be configured to deliver a plication maintenance device to the patient's abdominal cavity. In some embodiments, the kits include a delivery device that is configured to deploy the plication maintenance device. In some instances, the kits include a plication maintenance device preloaded on a delivery device, such that the kits includes a system made up of an implantable plication maintenance device in a constrained state on a delivery device. In some embodiments, the kits include a delivery device comprising a sheath, as described above, which is configured to cover or encase the plication maintenance device. In some embodiments, the kits include one or more devices configured to create an opening in a patient's abdominal wall, such as a trocar. In some instances, the kits may include a magnetic component, such as a magnet or a material that is attracted to a magnet.

The instructions for using the devices as discussed above are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub-packaging) etc. In other embodiments, the instructions are present as an electronic storage data file present on a suitable computer readable storage medium, e.g., CD-ROM, diskette, etc. The instructions may take any form, including complete instructions for how to use the device or as a website address with which instructions posted on the world wide web may be accessed.

The following examples are offered by way of illustration and not by way of limitation.

### EXPERIMENTAL

### Example 1

### Gastric Plication Procedure

After informed consent, a patient is given general anesthesia with endo-tracheal intubation. The abdomen is sterilely-prepared and draped in the usual fashion. Pneumoperitoneum is established after introducing a Veres needle and a 5-10 mm trocar is introduced into the peritoneal cavity. Under direct vision, two to three other trocars are placed. The short gastric vessels are then sealed and divided using mechanical or electromechanical sealers. The entire greater curvature of the stomach is mobilized from the angle of His cephalad to 5-7 cm proximal to the pylorus.

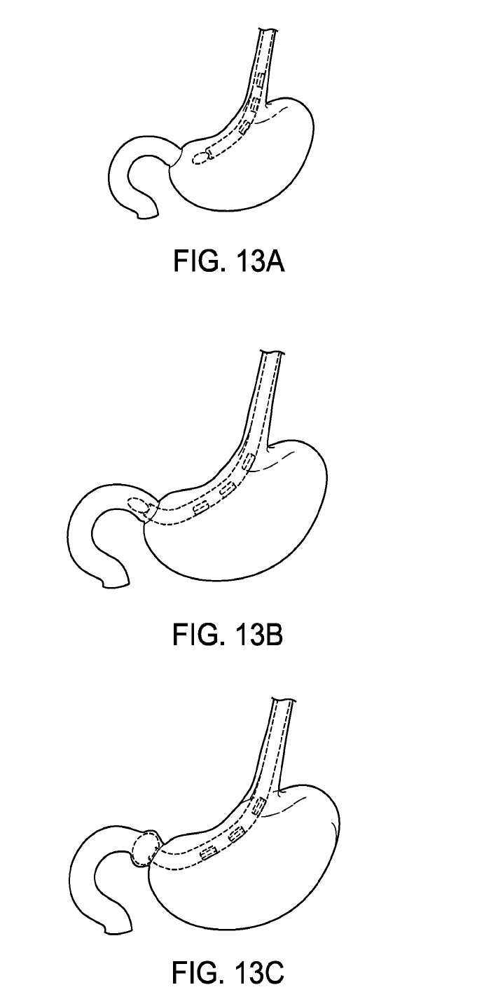

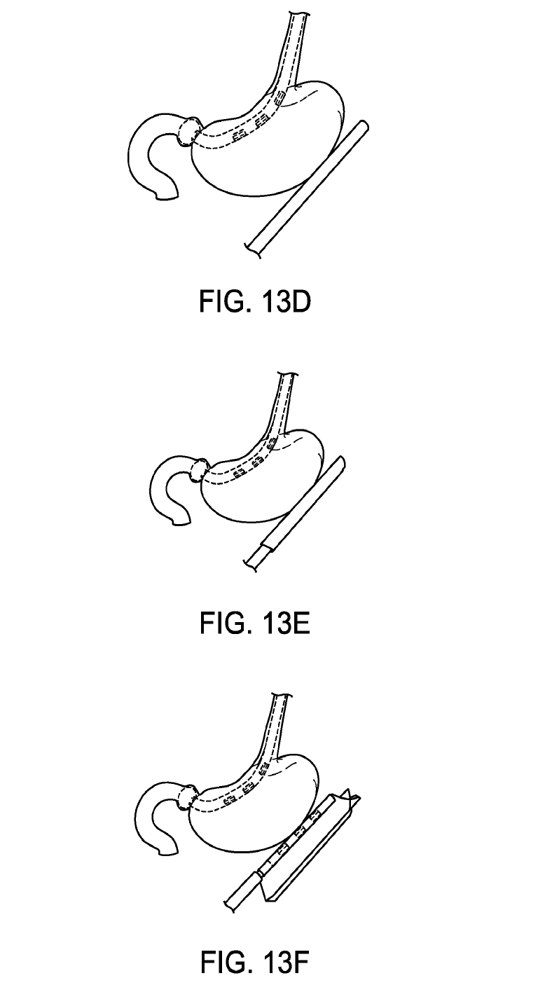

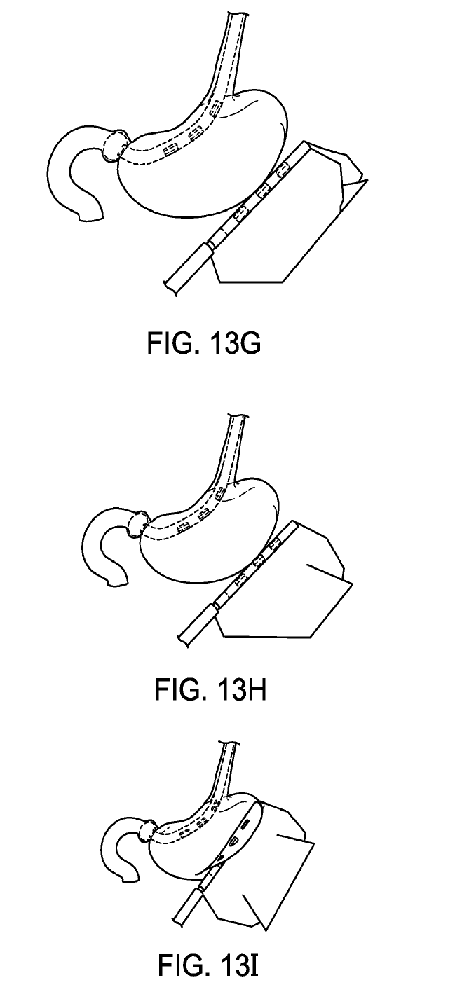

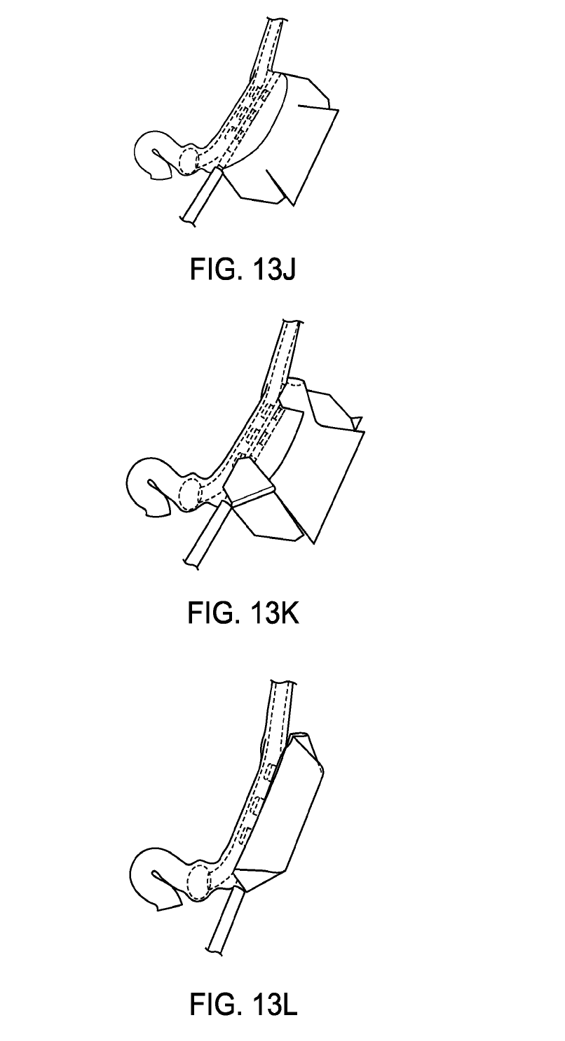

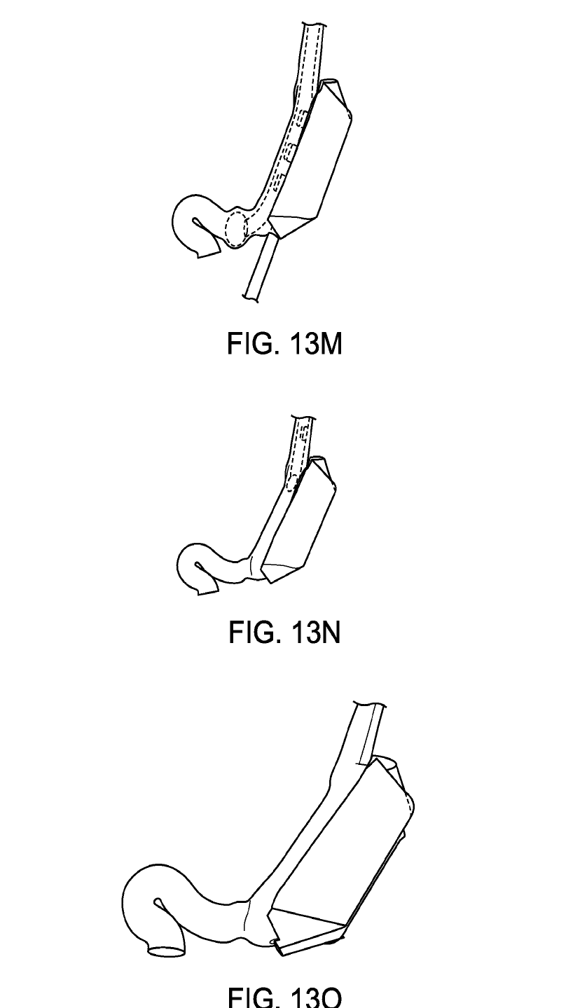

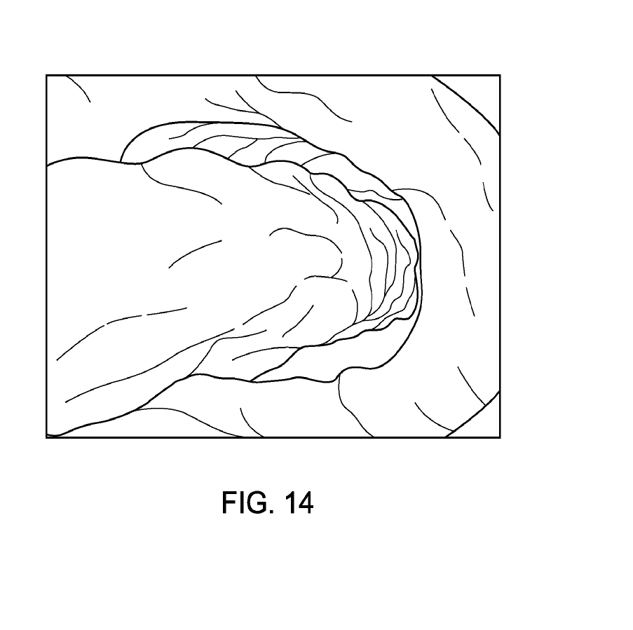

An endo-gastric tube including a magnetic component is introduced via the oral-esophageal path and advanced beyond the pylorus, as shown in FIGS. 13A and 13B. The distal balloon of the tube is insufflated in the proximal duodenum and fixed into position, as shown in FIG. 13C. Via one of the laparoscopic ports, a delivery device (which will serve as both a plication device and deployment device) carrying a plication maintenance device is introduced into the patient's abdominal cavity and placed proximal to the serosal surface of the stomach, as shown in FIG. 13D. The protective sheath is then retracted, as shown in FIG. 13E. Next, the plication maintenance device opened, as shown in FIGS. 13F to 13H. The greater curvature of the stomach is then approximated to the lesser curvature of the stomach, infolding the stomach from the angle of His to just proximal to the pylorus, as shown in FIGS. 13I to 13J. The greater curvature of the stomach is now in close proximity to the magnetic component of the endo-gastric tube that has been fixed along the lesser curvature of the stomach. Intragastric suction may be applied to ensure good serosal to serosal apposition of the anterior and posterior walls of the stomach with the plication maintenance device sandwiched in between. Intermittent insufflation of the lumen of the stomach may also be necessary to ensure good lateral spread of the infolded stomach and to ensure good apposition of the serosal surfaces of the stomach to the plication maintenance device. Components of the plication maintenance device are then folded anteriorly and posteriorly to brace the non-opposing surfaces of the infolded stomach to ensure good volume restriction in the anterior-posterior planes, e.g., as shown in FIGS. 13K to 13L. Where desired, the maintenance device may be trimmed to size to the level of the endo-gastric tube. The rod component of the delivery device is then removed as shown in FIGS. 13M and 13N; the intraduodenal balloon is deflated, the endo-gastric tube is removed, hemostasis is confirmed and all wounds are closed, resulting in a stably plicated stomach as shown in FIG. 13O. A picture of the interior of a plicated stomach which may be produced using the above protocol is provided in FIG. 14.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An organ plication system, the system comprising:
a plication maintenance device configured to maintain a folded gastric configuration; and
a plication/deployment component.

2. The organ plication system according to claim 1, wherein the plication maintenance device comprises a planar component.

3. The organ plication system according to claim 2, wherein the plication maintenance device further comprises a tubular component positioned to one side of the planar component.

4. The organ plication system according to claim 2, wherein the plication maintenance device further comprises a securing component.

5. The organ plication system according to claim 1 4, wherein the plication maintenance device further comprises a tissue securing component on at least one surface thereof.

6. The organ plication system according to claim 1, wherein the plication/deployment component comprises an endo-gastric tube.

7. The organ plication system according to claim 6, wherein the plication/deployment component further comprises a delivery device.

8. The organ plication system according to claim 7, wherein the delivery device and endo-gastric tube each comprise magnetic components.

9. The system according to claim 7, wherein the delivery device comprises a rod-shaped component.

10. The system according to claim 7, wherein the delivery system comprises a sheath.

11. The system according to claim 6, wherein the endo-gastric tube comprises a distal inflatable balloon.

12. The system according to claim 6, wherein the endo-gastric tube comprises one or more aspiration ports.

13-15. (canceled)

16. A method of reducing stomach volume of a subject, the method comprising operatively coupling a plication maintenance device to a stomach in a manner sufficient to produce a gastric plication.

17. The method according to claim 16, wherein the method comprises employing a gastric plication system according to claim 1.

18. A kit for use in a gastric plication procedure, the kit comprising:
a plication maintenance device and a plication/deployment component according to claim 1.
